(19) United States
(12) Patent Application Publication
(10) Pub. No.: US 2005/0222173 A1
(43) Pub. Date: Oct. 6, 2005

(54) Triazolopyrimidines

(76) Inventors: Christiane Boie, Leichlingen (DE); Ralf Dunkel, Monheim (DE); Hans-Ludwig Elbe, Wuppertal (DE); Herbert Gayer, Monheim (DE); Olaf Gebauer, Koln (DE); Bernd-Wieland Kruger, Bergisch Gladbach (DE); Ulrich Heinemann, Leichlingen (DE); Arnd Voerste, Koln (DE); Oliver Guth, Leverkusen (DE); Ronald Ebbert, Monheim (DE); Ulrike Wachendorff-Neumann, Neuwied (DE); Astrid Mauler-Machnik, Leichlingen (DE)

Correspondence Address: BAYER CROPSCIENCE LP;Patent Department, 100 BAYER ROAD, PITTSBURGH, PA, 15205-9741, US

(21) Appl. No.: 10/508,402

(22) PCT Filed: Mar. 10, 2003
(86) PCT No.: PCT/EP03/02413

§ 371 Date: Mar. 22, 2005

(30) Foreign Application Priority Data
Mar. 22, 2002 (DE) 102 12 886.3

(51) Int. Cl. A61K 31/519; C07D 487/02

(52) U.S. Cl. 514/259.31; 544/256

(57) ABSTRACT

Novel triazolopyrimidines of the formula 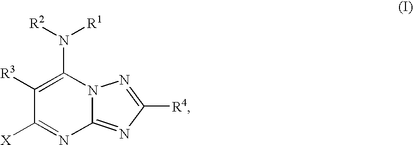 in which
R¹ represents amino, represents in each case optionally substituted alkyl, alkenyl, alkynyl, cycloalkyl, alkoxy, alkenyloxy, alkynyloxy, cycloalkyloxy, alkylamino, dialkylamino, alkenylamino, alkynylamino, cycloalkylamino, N-cycloalkyl-N-alkylamino, alkylideneamino or heterocyclyl, and
R² represents hydrogen or represents in each case optionally substituted alkyl, alkenyl, alkynyl or cycloalkyl, or
R¹ and R² together with the nitrogen atom to which they are attached form an optionally substituted heterocyclic ring,
R³ represents aryl which is optionally mono- to tetrasubstituted,
R⁴ represents halogen, cyano or represents in each case optionally substituted alkoxy or dialkylamino and X represents halogen, a process for preparing these novel substances and their use for controlling unwanted microorganisms.
   Novel intermediates of the formulae 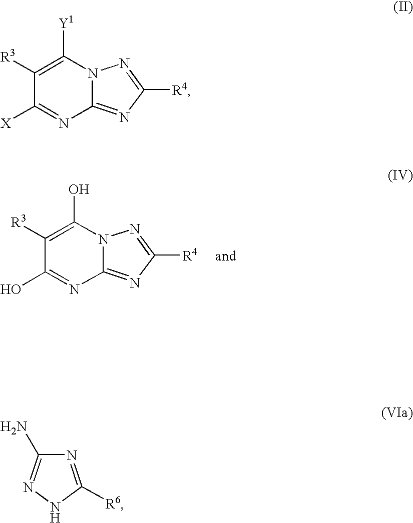 and processes for their preparation.

The invention relates to novel triazolopyrimidines, to a process for their preparation and to their use for controlling unwanted microorganisms.

The invention furthermore relates to novel intermediates and to processes for their preparation.

It is already known that certain triazolopyrimidines have fungicidal properties (cf. EP-A-0 550 113, WO 94-20501, EP-A 0 613 900, U.S. Pat. No. 5,612,345, EP-A 0 834 513, WO 9846607 and WO 9846608). However, in many cases the activity of these compounds is unsatisfactory.

This invention provides novel triazolopyrimidines of the formula 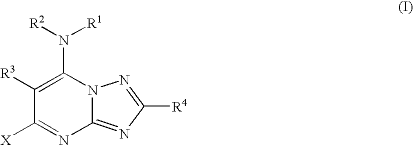
in which
R¹ represents amino, represents in each case optionally substituted alkyl, alkenyl, alkynyl, cycloalkyl, alkoxy, alkenyloxy, alkynyloxy, cycloalkyloxy, alkylamino, dialkylamino, alkenylamino, alkynylamino, cycloalkylamino, N-cycloalkyl-N-alkylamino, alkylideneamino or heterocyclyl, and
R² represents hydrogen or represents in each case optionally substituted alkyl, alkenyl, alkynyl or cycloalkyl, or
R¹ and R² together with the nitrogen atom to which they are attached form an optionally substituted heterocyclic ring,
R³ represents aryl which is optionally mono- to tetrasubstituted,
R⁴ represents halogen, cyano or represents in each case optionally substituted alkoxy or dialkylamino and
X represents halogen.

Furthermore, it has been found that novel triazolopyrimidines of the formula (I) are obtained when
a) dihalotriazolopyrimidines of the formula 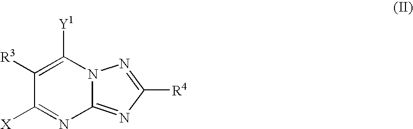
   in which
   R³, R⁴ and X are as defined above and
   Y¹ represents halogen
   are reacted with an amine of the formula 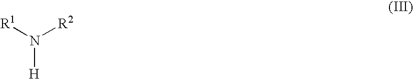
   in which
   R¹ and R² are as defined above,
   if appropriate in the presence of a diluent and if appropriate in the presence of an acid acceptor.

Finally, it has been found that the novel triazolopyrimidines of the formula (I) have very good activity against harmful organisms, and in particular strong fungicidal activity.

Surprisingly, triazolopyrimidines of the formula (I) have considerably better fungicidal activity than the constitutionally most similar prior-art substances of the same direction of action.

Particular meanings of the substituents or ranges of the formulae listed above and below are illustrated below.
R¹ preferably represents amino, represents alkyl, alkenyl, alkynyl, cycloalkyl, alkoxy, alkenyloxy, alkynyloxy, cycloalkyloxy, alkylamino, dialkylamino, alkenylamino, alkynylamino, cycloalkylamino, N-cycloalkyl-N-alkylamino or alkylideneamino having in each case 1 to 6 carbon atoms in the alkyl chains, 2 to 6 carbon atoms in the alkenyl and alkynyl chains, 3 to 7 carbon atoms in the cycloalkyl radicals or 2 to 6 carbon atoms in the case of alkylideneamino, each of which radicals is optionally substituted by 1 to 9 halogen atoms, hydroxyl, alkoxy, carbalkoxy, dialkylamino, cycloalkyl, cyano, phenyl or heterocyclyl, or represents heterocyclyl having 5 or 6 ring members.

The phenyl and heterocyclyl radicals mentioned may for their part be mono- or polysubstituted by identical or different substituents.

Preferred substituents for heterocyclyl are:
halogen, phenyl;
in each case straight-chain or branched alkyl, alkoxy or alkylthio having in each case 1 to 6 carbon atoms;
in each case, straight-chain or branched haloalkyl, haloalkoxy, haloalkylthio having in each case 1 to 6 carbon atoms and 1 to 13 identical or different halogen atoms.

Preferred substituents for phenyl are:
halogen, cyano, nitro, amino, hydroxyl, formyl, carboxyl, carbamoyl, thiocarbamoyl;
in each case straight-chain or branched alkyl, alkoxy, alkylthio, alkylsulphinyl or alkylsulphonyl having in each case 1 to 6 carbon atoms;
in each case straight-chain or branched alkenyl or alkenyloxy having in each case 2 to 6 carbon atoms;
in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
in each case straight-chain or branched haloalkenyl or haloalkenyloxy having in each case 2 to 6 carbon atoms and 1 to 11 identical or different halogen atoms;
in each case straight-chain or branched alkylamino, dialkylamino, alkylcarbonyl, alkylcarbonyloxy, alkoxycarbonyl, alkylsulphonyloxy, hydroximinoalkyl or alkoximinoalkyl having in each case 1 to 6 carbon atoms in the individual alkyl moieties;
in each case doubly attached alkylene or dioxyalkylene having in each case 1 to 6 carbon atoms and being in each case optionally mono- or polysubstituted by identical or different substituents from the group consisting of halogen, straight-chain or branched alkyl having 1 to 4 carbon atoms and straight-chain or branched haloalkyl having 1 to 4 carbon atoms and 1 to 9 identical or different halogen atoms; or
cycloalkyl having 3 to 6 carbon atoms.
R² preferably represents hydrogen or represents optionally halogen- or C₃-C₆-cycloalkyl-substituted C₁-C₄-alkyl, C₂-C₄-alkenyl, C₂-C₄-alkynyl or C₃-C₆-cycloalkyl.
R¹ and R² also preferably together with the nitrogen atom to which they are attached represent a 3- to 6-membered heterocyclic ring which is optionally substituted by halogen, hydroxyl, alkyl having 1 to 4 carbon atoms or haloalkyl having 1 to 4 carbon atoms and 1 to 9 halogen atoms.
R³ preferably represents phenyl which is optionally mono- to tetrasubstituted, preferred possible substituents being the following:
   halogen, cyano, nitro, amino, hydroxyl, formyl, carboxy, carbamoyl, thiocarbamoyl;
   in each case straight-chain or branched alkyl, alkoxy, alkylthio, alkylsulphinyl or alkylsulphonyl having in each case 1 to 6 carbon atoms;
   in each case straight-chain or branched alkenyl or alkenyloxy having in each case 2 to 6 carbon atoms;
   in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
   in each case straight-chain or branched haloalkenyl or haloalkenyloxy having in each case 2 to 6 carbon atoms and 1 to 13 identical or different halogen atoms;
   in each case straight-chain or branched alkylamino, dialkylamino, alkylcarbonyl, alkylcarbonyloxy, alkoxycarbonyl, alkylsulphonyloxy, hydroximinoalkyl or alkoximinoalkyl having in each case 1 to 6 carbon atoms in the individual alkyl moieties;
   in each case doubly attached alkylene or dioxyalkylene having in each case 1 to 6 carbon atoms and being in each case optionally mono- or polysubstituted by identical or different substituents from the group consisting of halogen, straight-chain or branched alkyl having 1 to 4 carbon atoms and straight-chain or branched haloalkyl having 1 to 4 carbon atoms and 1 to 9 identical or different halogen atoms, or
   cycloalkyl having 3 to 6 carbon atoms.
R⁴ preferably represents halogen, cyano or represents alkoxy or dialkylamino having in each case 1 to 6 carbon atoms in the alkyl chains and being in each case optionally substituted by 1 to 13 halogen atoms.
X preferably represents fluorine, chlorine or bromine.
R¹ particularly preferably represents amino or represents alkyl having 1 to 6 carbon atoms, where the alkyl radicals may be mono- to trisubstituted by fluorine, chlorine, hydroxyl, methoxy, dimethylamino, diethylamino, cyclopropyl, cyclobutyl, cyclopentyl, cyclohexyl, dioxolan-2-yl, 2-furyl, 2-tetrahydrofuryl, methoxycarbonyl, ethoxycarbonyl, phenyl and/or phenoxy, or
R¹ particularly preferably represents alkenyl having 3 to 6 carbon atoms or alkynyl having 3 to 6 carbon atoms, or
R¹ particularly preferably represents in each case optionally methyl-substituted cyclopropyl, cyclobutyl, cyclopentyl, cyclohexyl, pyrrolidinyl, piperidinyl, morpholinyl, thiomorpholinyl, piperazinyl, or
   represents in each case optionally fluorine-, hydroxyl-, methoxy-, dimethylamino-, cyclopropyl-, cyclobutyl-, cyclopentyl- or cyclohexyl-substituted methylamino, ethylamino, n- or i-propylamino, n-, i-, s- or t-butylamino, dimethylamino, 1-methoxycarbonyl-1-methylamino, methoxy, ethoxy or
   represents in each case optionally substituted benzyloxy, pyridylmethyloxy or thiazolylmethoxy.

The pyridyl and thiazolyl radicals mentioned may for their part also preferably be substituted further. Suitable substituents for pyridyl and thiazolyl are, preferably: fluorine, chlorine, bromine, methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, difluoromethoxy, trifluoromethoxy, difluorochloromethoxy, trifluoroethoxy, difluoromethylthio, difluorochloromethylthio, dichlorofluoromethylthio, trifluoromethylthio, phenyl.

Suitable substituents for phenyl and benzyl are, preferably:
fluorine, chlorine, bromine, cyano, nitro, amino, hydroxyl, formyl, carboxyl, carbamoyl, thiocarbamoyl, methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl or ethylsulphonyl, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl or trifluoromethylsulphonyl, methylamino, ethylamino, n- or i-propylamino, dimethylamino, diethylamino, acetyl, propionyl, acetyloxy, methoxycarbonyl, ethoxycarbonyl, methylsulphonyloxy, ethylsulphonyloxy, hydroximinomethyl, hydroximinoethyl, methoximinomethyl, ethoximinomethyl, methoximinoethyl or ethoximinoethyl,
in each case doubly attached trimethylene (propane-1,3-diyl), methylenedioxy or ethylenedioxy, each of which is optionally mono- or polysubstituted by identical or different substituents from the group consisting of fluorine, chlorine, methyl, trifluoromethyl, ethyl, n- and i-propyl,
cyclopropyl, cyclobutyl, cyclopentyl or cyclohexyl.
R² particularly preferably represents hydrogen, methyl, ethyl, n- or i-propyl, n-, i-, s- or t-butyl, allyl, propargyl, 2,2,2-trifluoroethyl, 1-(1,1,1-trifluoromethyl)ethyl, cyclopropylmethyl, cyclobutylmethyl, cyclopentylmethyl or cyclohexylmethyl.
R¹ and R² also particularly preferably together with the nitrogen atom to which they are attached represent in each case optionally fluorine-, hydroxyl-, methyl-, ethyl- or trifluoromethyl-substituted pyrrolidinyl, piperidinyl, morpholinyl, thiomorpholinyl, piperazinyl, 3,6-dihydro-1(2H)-pyridinyl or tetrahydro-1(2H)-pyridazinyl, or represent radicals of the formula 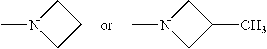
R³ particularly preferably represents mono- to tetrasubstituted phenyl. Possible substituents which are particularly preferred are the following:
   fluorine, chlorine, bromine, cyano, nitro, formyl, methyl, ethyl, n- or i-propyl, n-, i-, s- or t-butyl, allyl, propargyl, methoxy, ethoxy, n- or i-propoxy, methylthio, ethylthio, n- or i-propylthio, methylsulphinyl, ethylsulphinyl, methylsulphonyl or ethylsulphonyl, allyloxy, propargyloxy, trifluoromethyl, trifluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, trichloroethynyloxy, trifluoroethynyloxy, chloroallyloxy, iodopropargyloxy, methylamino, ethylamino, n- or i-propylamino, dimethylamino, diethylamino, acetyl, propionyl, acetyloxy, methoxycarbonyl, ethoxycarbonyl, hydroximinomethyl, hydroximinoethyl, methoximinomethyl, ethoximinomethyl, methoximinoethyl or ethoximinoethyl,
   in each case doubly attached trimethylene (propane-1,3-diyl), methylenedioxy or ethylenedioxy, each of which is optionally mono- or polysubstituted by identical or different substituents from the group consisting of fluorine, chlorine, methyl, trifluoromethyl, ethyl, n- or i-propyl,
   cyclopropyl, cyclobutyl, cyclopentyl or cyclohexyl.
R⁴ particularly preferably represents fluorine, chlorine, bromine, cyano, methoxy, ethoxy, n- or i-propoxy, n-, i-, s- or t-butoxy, trifluoromethoxy, trifluoroethoxy, dimethylamino, ethylmethylamino or diethylamino.
X particularly preferably represents fluorine or chlorine.

Very particular preference is given to triazolopyrimidines of the formula (I), in which
R¹ represents amino, methyl, ethyl, n-propyl, i-propyl, n-butyl, i-butyl, s-butyl, t-butyl, 1,2-dimethylpropyl, 2,2-dimethylpropyl, n-pentyl, 2-methylbutyl, 1,3-dimethylbutyl, 2-methoxyethyl, 2-hydroxyethyl, 1-trifluoromethylethyl, 2,2,2-trifluoroethyl, 1,1,1-trifluoro-2-methylprop-2-yl, methoxy, cyclopropylmethyl, 1-cyclohexylethyl, 2-dimethylaminoethyl, ethoxycarbonylmethyl, dioxolan-2-ylmethyl, 2-furylmethyl, 2-tetrahydrofurylmethyl, methylamino, ethylamino, n-propylamino, 1-cyclopropylethylamino, 2,2,3,3-tetrafluoropropylamino, N-methyl-N-methoxycarbonylamino, morpholinyl, cyclopropyl, cyclopentyl, cyclohexyl, allyl, 2-methylallyl or propargyl,
R² represents hydrogen, methyl, ethyl, n-propyl, i-propyl, n-butyl, i-butyl, s-butyl, t-butyl, allyl, propargyl, 2,2,2-trifluoroethyl, cyclopropylmethyl or cyclopentylmethyl, or
R¹ and R² together with the nitrogen atom to which they are attached represent a radical of the formula 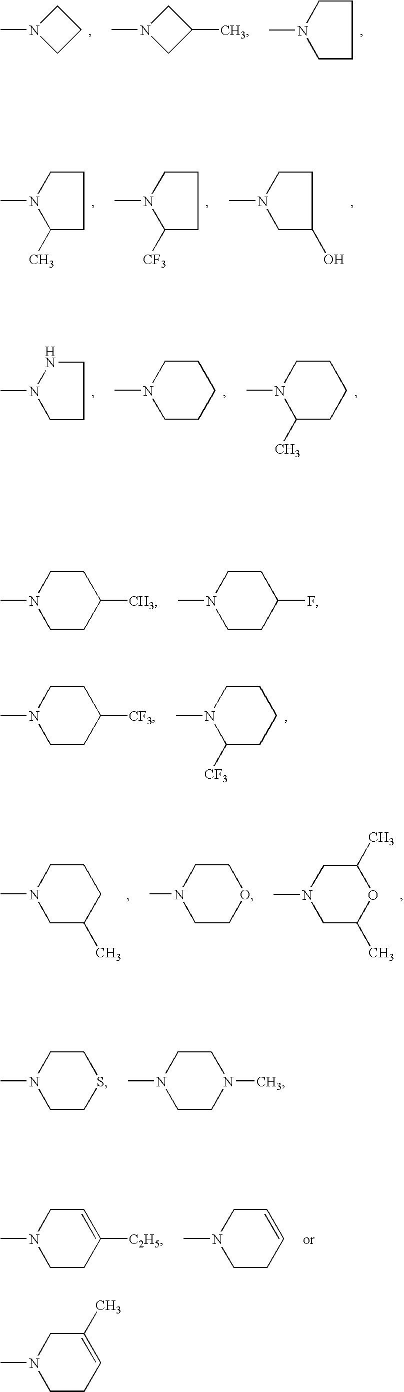
R³ represents a radical of the formula 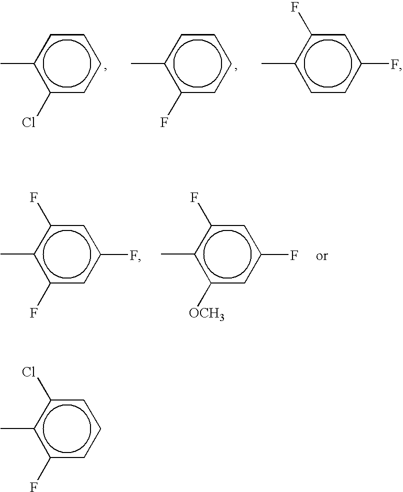
R⁴ represents fluorine, chlorine, methoxy or dimethylamino and
X represents fluorine or chlorine.

The general or preferred radical definitions given above apply both to the end products of the formula (I) and, correspondingly, to the starting materials or intermediates required in each case for the preparation.

The radical definitions mentioned above can be combined with one another as desired. Moreover, individual definitions may not apply.

Using, as starting materials, 2,5,7-trichloro-6-(2-chlorophenyl)[1,2,4]triazolo[1,5a]-pyrimidine and isopropylamine, the course of the process (a) according to the invention can be illustrated by the formula scheme below.

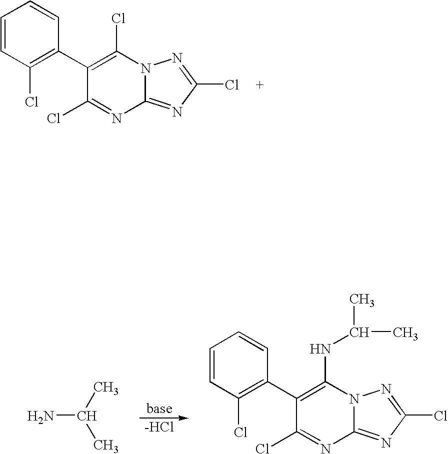

The formula (II) provides a general definition of the dihalotriazolopyrimidines required as starting materials for carrying out the process (a) according to the invention. In this formula (II), R³, R⁴ and X preferably or in particular have those meanings which have already been mentioned in connection with the description of the compounds of the formula (I) according to the invention as being preferred or being particularly preferred for R³, R⁴ and X. Y¹ preferably represents fluorine, chlorine or bromine, in particular fluorine or chlorine.

The dihalotriazolopyrimidines of the formula (II) are novel and also form part of the subject-matter of the present application. These compounds, too, are suitable for controlling unwanted microorganisms.

They are obtained when
(b) dihydroxytriazolopyrimidines of the formula 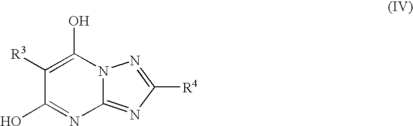
   in which
R³ and R⁴ are as defined above,
   are reacted with a halogenating agent, such as, for example, phosphorus trichloride, phosphorus tribromide, phosphorus pentachloride, phosphorus oxychloride, thionyl chloride, thionyl bromide or mixtures thereof, if appropriate in the presence of a diluent, such as, for example, chlorobenzene. Other possible diluents are the halogenating agent itself, such as phosphorus oxychloride, or a mixture of halogenating agents.

The formula (IV) provides a general definition of the dihydroxytriazolopyrimidines required as starting materials for carrying out the process (b) according to the invention. In this formula, R³ and R⁴ preferably or in particular have those meanings which have already been mentioned above, in connection with the description of the compounds of the formula (I) according to the invention as being preferred or as being particularly preferred for R³ and R⁴.

The dihalotriazolopyrimidines of the formula (IV) are novel and also form part of the subject-matter of the present application. These compounds, too, are suitable for controlling unwanted microorganisms.

They are obtained when
(c) arylmalonic esters of the formula 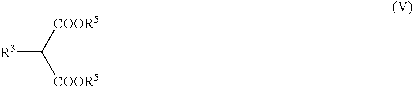
   in which
R³ is as defined above and
R⁵ represents alkyl having 1 to 4 carbon atoms
   are reacted with an aminotriazole of the formula 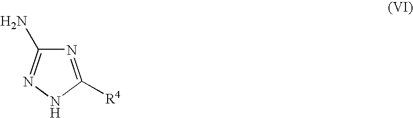
   in which
R⁴ is as defined above,
   if appropriate in the presence of a diluent, such as, for example, an alcohol, and if appropriate in the presence of a base, preferably a tertiary amine, such as, for example, tributylamine. The amine used as base can simultaneously serve as diluent.

The formula (V) provides a general definition of the arylmalonic esters required as starting materials for carrying out the process (c) according to the invention. In this formula, R³ preferably or in particular has that meaning which has already been mentioned in connection with the description of the compounds of the formula (I) according to the invention as being preferred or as being particularly preferred for R³. R⁵ preferably represents methyl or ethyl.

The arylmalonic esters of the formula (V) are known or can be prepared by known methods (cf. U.S. Pat. No. 6,156,925).

The formula (VI) provides a general definition of the aminotriazoles furthermore required as starting materials for carrying out the process (c) according to the invention. In this formula, R⁴ preferably or in particular has that meaning which has already been given in connection with the description of the compounds of the formula (I) according to the invention as being preferred or as being particularly preferred for R⁴.

The aminotriazoles of the formula (VI) are known chemicals for synthesis or can be prepared by known methods (cf. Russian Journal of Organic Chemistry, Vol. 29, No. 11, 1993, pages 1942-1943).

The aminotriazoles of the formula 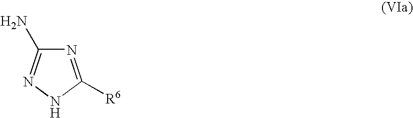
in which
R⁶ represents cyano or bromine,
   are novel and form part of the subject-matter of the present application. These compounds, too, are suitable for controlling unwanted microorganisms.

They are obtained by initially diazotizing diaminotriazole of the formula 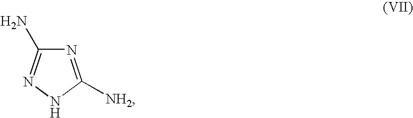
and then reacting them with a brominating agent, such as, for example, hydrogen bromide, or a cyanating agent such as, for example, hydrogen cyanide, if appropriate in the presence of a diluent and if appropriate in the presence of further reaction auxiliaries.

The halogenating agents furthermore required as starting materials for carrying out the process (b) according to the invention are generally known laboratory chemicals.

The formula (III) provides a general definition of the amines furthermore required as starting materials for carrying out the process (a) according to the invention. In this formula (III), R¹ and R² preferably or in particular have those meanings which have already been mentioned in connection with the description of the compounds of the formula (I) according to the invention as being preferred or as being particularly preferred for R¹ and R².

The amines of the formula (III) are generally customary laboratory chemicals or can be obtained by known methods.

Suitable eluents for carrying out the process (a) according to the invention are all inert organic solvents. Preference is given to using aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; sulphones, such as sulpholane.

The process (a) according to the invention is, if appropriate, carried out in the presence of a suitable acid acceptor. Suitable acid acceptors are, preferably, ammonia or tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process (a) according to the invention, the reaction temperatures can be varied within a relatively wide range. In general, the process is carried out at temperatures of from 0° C. to 150° C., preferably at temperatures of from 0° C. to 80° C.

When carrying out the processes (b) and (c) according to the invention, it is also possible to vary the reaction temperatures within this range.

For carrying out the process (a) according to the invention for preparing the compounds of the formula (I), in general from 0.5 to 10 mol, preferably from 0.8 to 2 mol, of amine of the formula (III) are employed per mole of dihalotriazolopyrimidine of the formula (II).

The processes according to the invention are generally carried out under atmospheric pressure. However, it is also possible to operate under elevated or reduced pressure—in general between 0.1 bar and 10 bar.

The substances according to the invention have potent microbicidal activity and can be employed for controlling unwanted microorganisms, such as fungi and bacteria, in crop protection and in the protection of material.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:
Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such as, for example, Pythium ultimum;
Phytophthora species, such as, for example, Phytophthora infestans;
Pseudoperonospora species, such as, for example, Pseudoperonospora humuli or Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea (conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasakii;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and
Pseudocercosporella species, such as, for example, Pseudocercosporella herpotrichoides.

The active compounds according to the invention also have very good fortifying action in plants. Accordingly, they can be used for mobilizing the plants own defences against attack by unwanted microorganisms.

In the present context, plant-fortifying (resistance-inducing) substances are to be understood as meaning those substances which are capable of stimulating the defence system of plants such that, when the treated plants are subsequently inoculated with unwanted microorganisms, they show substantial resistance against these microorganisms.

In the present case, unwanted microorganisms are to be understood as meaning phytopathogenic fungi, bacteria and viruses. Accordingly, the substances according to the invention can be used to protect plants for a certain period after the treatment against attack by the pathogens mentioned. The period for which protection is provided generally extends over 1 to 10 days, preferably 1 to 7 days, after the treatment of the plants with the active compounds.

The fact that the active compounds are well tolerated by plants at the concentrations required for controlling plant diseases permits treatment of above-ground parts of plants, of propagation stock and seeds, and of the soil.

The active compounds according to the invention can be used with particularly good results for controlling diseases in viticulture and in the cultivation of fruits and vegetables such as, for example, against Venturia and Podosphaera speces.

The active compounds according to the invention are also suitable for increasing the yield of crops. In addition, they show reduced toxicity and are well tolerated by plants.

At certain concentrations and application rates, the active compounds according to the invention can also be used as herbicides, for influencing plant growth and for controlling animal pests. They can also be used as intermediates and precursors for the synthesis of further active compounds.

The active compounds according to the invention can be used to treat all plants and parts of plants. By plants are understood here all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant varieties which can or cannot be protected by varietal property rights. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment of the plants and parts of plants with the active compounds according to the invention is carried out directly or by action on their surroundings, habitat or storage space, according to customary treatment methods, for example by dipping, spraying, evaporating, atomizing, broadcasting, spreading-on and, in the case of propagation material, in particular in the case of seeds, furthermore by single- or multi-layer coating.

In the protection of materials, the compounds according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes), and against slime organisms and algae.

Microorganisms of the following genera may be mentioned as examples:
Altemaria, such as Altemaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted to the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as, finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can be used as such or in their formulations, also in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Examples of suitable mixing components are the following:

Fungicides:

aldimorph, ampropylfos, ampropylfos potassium, andoprim, anilazine, azaconazole, azoxystrobin,
benalaxyl, benodanil, benomyl, benzamacril, benzamacril-isobutyl, bialaphos, binapacryl, biphenyl, bitertanol, blasticidin-S, bromuconazole, bupirimate, buthiobate,
calcium polysulphide, carpropamide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, carpropamide,
debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,
edifenphos, epoxiconazole, etaconazole, ethirimol, etridiazole,
famoxadon, fenapanil, fenarimol, fenbuconazole, fenfuram, fenhexamide, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulphamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, fluoxastrobin,
guazatine,
hexachlorobenzene, hexaconazole, hymexazole,
imazalil, imibenconazole, iminoctadine, iminoctadine albesilate, iminoctadine triacetate, iodocarb, ipconazole, iprobenfos (IBP), iprodione, iprovalicarb, irumamycin, isoprothiolane, isovaledione,
kasugamycin, kresoxim-methyl, copper preparations, such as: copper hydroxide, copper naphthenate, copper oxychloride, copper sulphate, copper oxide, oxine-copper and Bordeaux mixture,
mancopper, mancozeb, maneb, meferimzone, mepanipyrim, mepronil, metalaxyl, metconazole, methasulphocarb, methfuroxam, metiram, metomeclam, metsulphovax, mildiomycin, myclobutanil, myclozolin,
nickel dimethyldithiocarbamate, nitrothal-isopropyl, nuarimol,
ofurace, oxadixyl, oxamocarb, oxolinic acid, oxycarboxim, oxyfenthiin,
paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur, prothioconazole,
quinconazole, quintozene (PCNB), quinoxyfen,
sulphur and sulphur preparations, spiroxamines,
tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole,
uniconazole,
validamycin A, vinclozolin, viniconazole,
zarilamide, zineb, ziram and also
Dagger G,
OK-8705,
OK-8801,
α-(1,1-dimethylethyl)-β-(2-phenoxyethyl)-1H-1,2,4-triazole-1-ethanol,
α-(2,4-dichlorophenyl)-β-fluoro-β-propyl-1H-1,2,4-triazole-1-ethanol,
α-(2,4-dichlorophenyl)-β-methoxy-α-methyl-1H-1,2,4-triazole-1-ethanol,
α-(5-methyl-1,3-dioxan-5-yl)-β-[[4-(trifluoromethyl)phenyl]methylene]-1H-1,2,4-triazole-1-ethanol,
(5RS,6RS)-6-hydroxy-2,2,7,7-tetramethyl-5-(1H-1,2,4-triazol-1-yl)-3-octanone,
(E)-α-(methoxyimino)-N-methyl-2-phenoxy-phenylacetamide,
1-(2,4-dichlorophenyl)-2-(1H-1,2,4-triazol-1-yl)ethanone O-(phenylmethyl)oxime,
1-(2-methyl-1-naphthalenyl)-1H-pyrrole-2,5-dione,
1-(3,5-dichlorophenyl)-3-(2-propenyl)-2,5-pyrrolidinedione,
1-[(diiodomethyl)-sulphonyl]-4-methylbenzene,
1-[[2-(2,4-dichlorophenyl)-1,3-dioxolan-2-yl]methyl]-1H-imidazole,
1-[[2-(4-chlorophenyl)-3-phenyloxiranyl]methyl]-1H-1,2,4-triazole,
1-[1-[2-[(2,4-dichlorophenyl)methoxy]phenyl]ethenyl]-1H-imidazole,
1-methyl-5-nonyl-2-(phenylmethyl)-3-pyrrolidinole,
2′,6′-dibromo-2-methyl-4′-trifluoromethoxy-4′-trifluoromethyl-1,3-thiazole-5-carboxanilide,
2,6-dichloro-5-(methylthio)-4-pyrimidinyl-thiocyanate,
2,6-dichloro-N-(4-trifluoromethylbenzyl)benzamide,
2,6-dichloro-N-[[4-(trifluoromethyl)phenyl]methyl]benzamide,
2-(2,3,3-triiodo-2-propenyl)-2H-tetrazole,
2-[(1-methylethyl)-sulphonyl]-5-(trichloromethyl)-1,3,4-thiadiazole,
2-[[6-deoxy-4-O-(4-O-methyl-β-D-glycopyranosyl)-α-D-glucopyranosyl]amino]-4-methoxy-1H-pyrrolo[2,3-d]pyrimidine-5-carbonitrile,
2-aminobutane,
2-bromo-2-(bromomethyl)pentanedinitrile,
2-chloro-N-(2,3-dihydro-1,1,3-trimethyl-1H-inden-4-yl)-3-pyridinecarboxamide,
2-chloro-N-(2,6-dimethylphenyl)-N-(isothiocyanatomethyl)acetamide,
2-phenylphenol (OPP),
3,4-dichloro-1-[4-(difluoromethoxy)phenyl]-1H-pyrrole-2,5-dione,
3,5-dichloro-N-[cyano[(1-methyl-2-propynyl)oxy]methyl]benzamide,
3-(1,1-dimethylpropyl-1-oxo-1H-indene-2-carbonitrile,
3-[2-(4-chlorophenyl)-5-ethoxy-3-isoxazolidinyl]pyridine,
4-chloro-2-cyano-N,N-dimethyl-5-(4-methylphenyl)-1H-imidazole-1-sulphonamide,
4-methyltetrazolo[1,5-a]quinazolin-5(4H)-one,
8-hydroxyquinoline sulphate,
9H-xanthene-2-[(phenylamino)carbonyl]-9-carboxylic hydrazide,
bis-(1-methylethyl)-3-methyl-4-[(3-methylbenzoyl)oxy]-2,5-thiophenedicarboxylate,
cis-1-(4-chlorophenyl)-2-(1H-1,2,4-triazol-1-yl)cycloheptanol,
cis-4-[3-[4-(1,1-dimethylpropyl)phenyl-2-methylpropyl]-2,6-dimethylmorpholine-hydrochloride,
ethyl[(4-chlorophenyl)azo]cyanoacetate,
potassium hydrogen carbonate,
methanetetrathiol sodium salt,
methyl 1-(2,3-dihydro-2,2-dimethyl-1H-inden-1-yl)-1H-imidazole-5-carboxylate,
methyl N-(2,6-dimethylphenyl)-N-(5-isoxazolylcarbonyl)-DL-alaninate,
methyl N-(chloroacetyl)-N-(2,6-dimethylphenyl)-DL-alaninate,
N-(2,6-dimethylphenyl)-2-methoxy-N-(tetrahydro-2-oxo-3-furanyl)acetamide,
N-(2,6-dimethylphenyl)-2-methoxy-N-(tetrahydro-2-oxo-3-thienyl)acetamide,
N-(2-chloro-4-nitrophenyl)₄-methyl-3-nitrobenzenesulphonamide,
N-(4-cyclohexylphenyl) 1,4,5,6-tetrahydro-2-pyrimidineamine,
N-(4-hexylphenyl)-1,4,5,6-tetrahydro-2-pyrimidineamine,
N-(5-chloro-2-methylphenyl)-2-methoxy-N-(2-oxo-3-oxazolidinyl)acetamide,
N-(6-methoxy-3-pyridinyl)cyclopropanecarboxamide,
N-[2,2,2-trichloro-1-[(chloroacetyl)amino]ethyl]benzamide,
N-[3-chloro-4,5-bis-(2-propinyloxy)phenyl]-N′-methoxymethanimidamide,
N-formyl-N-hydroxy-DL-alanine sodium salt,
O,O-diethyl[2-(dipropylamino)-2-oxoethyl]ethylphosphoramidothioate,
O-methyl S-phenyl phenylpropylphosphoramidothioate,
S-methyl 1,2,3-benzothiadiazole-7-carbothioate,
spiro[2H]-1-benzopyrane-2,1′(3′H)-isobenzofuran]-3′-one,
4-[(3,4-dimethoxyphenyl)-3-(4-fluorophenyl)acryloyl]morpholine.
   Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.
   Insecticides/Acaricides/Nematicides:
abamectin, acephate, acetamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin,
Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,
cadusafos, carbaryl, carbofuran, carbophenothion, carbosulphan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cisresmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,
deltamethrin, demeton M, demeton S, demeton-5-methyl, diafenthiuron, diazinon, dichlorvos, dicofol, diflubenzuron, dimethoate, dimethylvinphos, diofenolan, disulphoton, docusat-sodium, dofenapyn,
eflusilanate, emamectin, empenthrin, endosulphan, Entomopfthora spp., esfenvalerate, ethiofencarb, ethion, ethoprophos, etofenprox, etoxazole, etrimfos,
fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,
granulosis viruses,
halofenozide, HCH, heptenophos, hexaflumuron, hexythiazox, hydroprene,
imidacloprid, indoxacarb, isazofos, isofenphos, isoxathion, ivermectin,
nuclear polyhedrosis viruses,
lambda-cyhalothrin, lufenuron,
malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,
naled, nitenpyram, nithiazine, novaluron,
omethoate, oxamyl, oxydemethon M,
Paecilomyces fumosoroseus, parathion A, parathion M, permethrin, phenthoate, phorat, phosalone, phosmet, phosphamidon, phoxim, pirimicarb, pirimiphos A, pirimiphos M, profenofos, promecarb, propargite, propoxur, prothiofos, prothoat, pymetrozine, pyraclofos, pyresmethrin, pyrethrum, pyridaben, pyridathion, pyrimidifen, pyriproxyfen,
quinalphos,
ribavirin,
salithion, sebufos, silafluofen, spinosad, spirodiclofen, sulphotep, sulprofos,
tau-fluvalinate, tebufenozide, tebufenpyrad, tebupirimiphos, teflubenzuron, tefluthrin, temephos, temivinphos, terbufos, tetrachlorvinphos, tetradifon thetacypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,
vamidothion, vaniliprole, Verticillium lecanii,
YI 5302,
Zeta-cypermethrin, Zolaprofos
(1R-cis)-[5-(phenylmethyl)-3-furanyl]methyl 3-[(dihydro-2-oxo-3(2H)-furanylidene)-methyl]-2,2-dimethylcyclopropanecarboxylate,
(3-phenoxyphenyl)methyl 2,2,3,3-tetramethylcyclopropanecarboxylate,
1-[(2-chloro-5-thiazolyl)methyl]tetrahydro-3,5-dimethyl-N-nitro-1,3,5-triazine-2(1H)-imine,
2-(2-chloro-6-fluorophenyl)-4-[4-(1,1-dimethylethyl)phenyl]4,5-dihydrooxazole,
2-(acetyloxy)-3-dodecyl-1,4-naphthalenedione,
2-chloro-N-[[[4-(1-phenylethoxy)phenyl]amino]carbonyl]benzamide,
2-chloro-N-[[[4-(2,2-dichloro-1,1-difluoroethoxy)phenyl]amino]carbonyl]-benzamide,
3-methylphenyl propylcarbamate,
4-[4-(4-ethoxyphenyl)-4-methylpentyl]-1-fluoro-2-phenoxybenzene,
4-chloro-2-(1,1-dimethylethyl)-5-[[2-(2,6-dimethyl-4-phenoxyphenoxy)ethyl]thio]-3(2H)-pyridazinone,
4-chloro-2-(2-chloro-2-methylpropyl)-5-[(6-iodo-3-pyridinyl)methoxy]-3(2H)-pyridazinone,
4-chloro-5-[(6-chloro-3-pyridinyl)methoxy]-2-(3,4-dichlorophenyl)-3(2H)-pyridazinone,
Bacillus thuringiensis strain EG-2348,
[2-benzoyl-1-(1,1-dimethylethyl)hydrazinobenzoic acid,
2,2-dimethyl-3-(2,4-dichlorophenyl)-2-oxo-1-oxaspiro[4.5]dec-3-en4-yl butanoate,
[3-[(6-chloro-3-pyridinyl)methyl]-2-thiazolidinylidene]cyanamide,
dihydro-2-(nitromethylene)-2H-1,3-thiazine-3(4H)carboxaldehyde,
ethyl[2-[[1,6-dihydro-6-oxo-1-(phenylmethyl)-4-pyridazinyl]oxy]ethyl]carbamate,
N-(3,4,4-trifluoro-1-oxo-3-butenyl)glycine,
N-(4-chlorophenyl)-3-[4-(difluoromethoxy)phenyl]4,5-dihydro-4-phenyl-1H-pyrazole-1-carboxamide,
N-[(2-chloro-5-thiazolyl)methyl]-N′-methyl-N″-nitroguanidine,
N-methyl-N′-(1-methyl-2-propenyl)-1,2-hydrazinedicarbothioamide,
N-methyl-N′-2-propenyl-1,2-hydrazinedicarbothioamide,
O,O-diethyl[2-(dipropylamino)-2-oxoethyl]ethylphosphoramidothioate,
N-cyanomethyl-4-trifluoromethylnicotinamide,
3,5-dichloro-1-(3,3-dichloro-2-propenyloxy)-4-[3-(5-trifluoromethylpyridine-2-yloxy)propoxy]benzene.

A mixture with other known active compounds, such as herbicides, or with fertilizers and growth regulators, is also possible.

In addition, the compounds of the formula (I) according to the invention also have very good antimycotic activity. They have a very broad antimycotic activity spectrum in particular against dermatophytes and yeasts, moulds and diphasic fungi, (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The list of these fungi by no means limits the mycotic spectrum covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10,000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10,000 g/ha, preferably between 1 and 5000 g/ha.

As already mentioned above, it is possible to treat all plants and their parts with active compounds according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased defence of the plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds of the formula (I) or the active compound mixtures according to the invnetion. The preferred ranges stated above for the active compounds and mixtures also apply to the treatment of these plants. Particular emphasis is given to the treatment of plants with the compounds or mixtures specifically mentioned in the present text.

The active compounds according to the invention can be employed with particularly good results for controlling diseases in viticulture and in the cultivation of fruits and vegetables, such as, for example, against Venturia and Podosphaera species.

The preparation and the use of the active compounds according to the invention are illustrated by the examples below.

### PREPARATION EXAMPLES

### Example 1

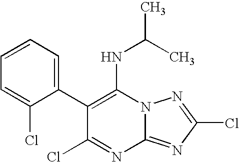 Process a)

0.33 g (1.0 mmol) of 2,5,7-trichloro-6-(2-chlorophenyl)[1,2,4]triazolo[1,5-a]pyrimidine is dissolved in 10 ml of dichloroethane. 0.12 g (2.0 mmol) of isopropylamine and 0.1 g of triethylamine are added to the solution. The mixture is stirred in a closed tube at 60° C. for 2 hours. After cooling, the solvent is removed under reduced pressure and the residue is purified by silica gel column chromatography (cyclohexane/ethyl acetate 1:1). This gives 0.11 g (18% of theory, purity 65%) of N-[2,5-dichloro-6-(2-chlorophenyl)[1,2,4]triazolo[1,5-a]pyrimidin-7-yl]-N-isopropylamine.

HPLC: logP=3.43

The compounds of the formula (I) listed in Table 1 below are also obtained analogously to Example 1 and in accordance with the statements in the general descriptions of the process.

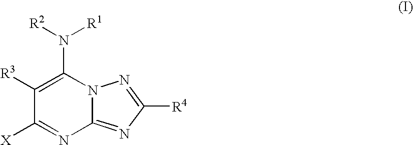

TABLE 1

| Ex. No. | R¹ | R² | R³ | R⁴ | X | log.P*) |
|---|---|---|---|---|---|---|
| 2 | 2,2,2-trifluoro-1-methylethyl | —H | 2,4,6-trifluorophenyl | —O—CH₃ | —Cl | 3.26 |
| 3 | 2,2,2-trifluoro-1-methylethyl | —H | 2,4,6-trifluorophenyl | —N(CH₃)₂ | —Cl | 3.52 |
| 4 | i-propylamino | —H | 2,4,6-trifluorophenyl | —N(CH₃)₂ | —Cl | 3.33 |
| 5 | —CH₂—CH₂—O—CH₂—CH₂— | | 2,4,6-trifluorophenyl | —Cl | —Cl | 2.99 |
| 6 | —CH₂—CH(CH₃)—O—CH(CH₃)—CH₂— | | 2,4,6-trifluorophenyl | —Cl | —Cl | 3.72 |
| 7 | —C₂H₅ | —H | 2,4,6-trifluorophenyl | —Cl | —Cl | 3.07 |
| 8 | —CH₂—CH₂—CH₂—CH₂— | | 2,4,6-trifluorophenyl | —Cl | —Cl | 3.46 |
| 9 | —C₂H₅ | —C₂H₅ | 2,4,6-trifluorophenyl | —Cl | —Cl | 3.85 |
| 10 | n-propyl | —H | 2,4,6-trifluorophenyl | —Cl | —Cl | 3.37 |
| 11 | cyclopentyl | —H | 2,4,6-trifluorophenyl | —Cl | —Cl | 3.9 |
| 12 | 2-methoxyethyl | —H | 2,4,6-trifluorophenyl | —Cl | —Cl | 2.91 |
| 13 | —CH₃ | —H | 2,4,6-trifluorophenyl | —Cl | —Cl | 2.67 |
| 14 | cyclopropyl | —H | 2,4,6-trifluorophenyl | —Cl | —Cl | 3.1 |
| 15 | —CH₂—CH₂—S—CH₂—CH₂— | | 2,4,6-trifluorophenyl | —Cl | —Cl | 3.68 |
| 16 | N-morpholinyl | —H | 2,4,6-trifluorophenyl | —Cl | —Cl | 2.74 |
| 17 | 1-cyclopropylethylamino | —H | 2-chlorophenyl | —Cl | —Cl | 3.55 |
| 18 | —NH—CH₂—CF₂—CHF₂ | —H | 2-chlorophenyl | —Cl | —Cl | 3.59 |
| 19 | cyclopropylmethyl | —H | 2-chlorophenyl | —Cl | —Cl | 3.55 |
| 20 | 1-cyclohexylethyl | —H | 2-chlorophenyl | —Cl | —Cl | |
| 21 | —CH₂CH₂OH | —H | 2-chlorophenyl | —Cl | —Cl | 2.18 |
| 22 | t-butyl | —H | 2-chlorophenyl | —Cl | —Cl | 3.99 |
| 23 | 1-cyclohexylethyl | —H | 2,4,6-trifluorophenyl | —Cl | —Cl | 4.87 |
| 24 | —CH₂CH₂OH | —H | 2,4,6-trifluorophenyl. | —Cl | —Cl | 2.23 |
| 25 | t-butyl | —H | 2,4,6-trifluorophenyl | —Cl | —Cl | 3.9 |
| 26 | —CH₃ | —CH₃ | 2-chlorophenyl | —Cl | —Cl | 3.18 |
| 27 | —CH₂—CH₂—O—CH₂—CH₂— | | 2-chlorophenyl | —Cl | —Cl | 3.03 |
| 28 | —CH₂—CH(CH₃)—O—CH(CH₃)—CH₂— | | 2-chlorophenyl | —Cl | —Cl | 3.81 |
| 29 | —C₂H₅ | —H | 2-chlorophenyl | —Cl | —Cl | 3.07 |
| 30 | —CH₂—CH₂—CH₂—CH₂— | | 2-chlorophenyl | —Cl | —Cl | 3.59 |
| 31 | —C₂H₅ | —C₂H₅ | 2-chlorophenyl | —Cl | —Cl | 3.99 |
| 32 | —CH₂—CH₂—CH₂—CH₂—CH₂— | | 2-chlorophenyl | —Cl | —Cl | 4.18 |
| 33 | n-propyl | —H | 2-chlorophenyl | —Cl | —Cl | 3.46 |
| 34 | cyclopentyl | —H | 2-chlorophenyl | —Cl | —Cl | 4.03 |
| 35 | 2-methoxyethyl | —H | 2-chlorophenyl | —Cl | —Cl | 2.91 |
| 36 | —CH₃ | —H | 2-chlorophenyl | —Cl | —Cl | 2.7 |
| 37 | cyclopropyl | —H | 2-chlorophenyl | —Cl | —Cl | 3.18 |
| 38 | —CH₂—CH₂—S—CH₂—CH₂— | | 2-chlorophenyl | —Cl | —Cl | 3.76 |
| 39 | N-morpholinyl | —H | 2-chlorophenyl | —Cl | —Cl | |
| 40 | dimethylamino | —H | 2-chlorophenyl | —Cl | —Cl | 3.18 |
| 41 | —N(CH₃)—COOCH₃ | —H | 2-chlorophenyl | —Cl | —Cl | 2.55 |
| 42 | cyclohexyl | —H | 2-chlorophenyl | —Cl | —Cl | 4.32 |
| 43 | -i-propyl | —H | 2,4,6-tnfluorophenyl | —Br | —Cl | 3.4 |
| 44 | 2,2,2-trifluoro-1-methylethyl | —H | 2,4,6-trifluorophenyl | —Br | —Cl | |
| 45 | -i-propyl | —H | 2-chlorophenyl | —Br | —Cl | 3.45 |
| 46 | —CH₂—CH₂—CH(CF₃)—CH₂—CH₂— | | 2,4,6-trifluorophenyl | —Cl | —Cl | 5 |
| 47 | -i-propyl | —H | 2,4,6-trifluorophenyl | —Cl | —Cl | 3.36 |
| 48 | —NH₂ | i-butyl | 2,4,6-trifluorophenyl | —Cl | —Cl | 3.67 |
| 49 | —CH₂—CH₂—O—CH₂—CH₂— | | 2,4,6-trifluorophenyl | —Br | —Cl | 3.03 |
| 50 | —CH₂—CH(CH₃)—O—CH(CH₃)—CH₂— | | 2,4,6-trifluorophenyl—Br | —Cl | 3.72 | |
| 51 | —C₂H₅ | —H | 2,4,6-trifluorophenyl | —Br | —Cl | 3.07 |
| 52 | —CH₂—CH₂—CH₂—CH₂— | | 2,4,6-trifluorophenyl | —Br | —Cl | 3.5 |
| 53 | —C₂H₅ | —C₂H₅ | 2,4,6-trifluorophenyl | —Br | —Cl | 3.9 |
| 54 | n-propyl | —H | 2,4,6-trifluorophenyl | —Br | —Cl | 3.41 |
| 55 | 2-methoxyethyl | —H | 2,4,6-trifluorophenyl | —Br | —Cl | 2.99 |
| 56 | cyclopropyl | —H | 2,4,6-trifluorophenyl | —Br | —Cl | 3.14 |
| 57 | N-morpholinyl | —H | 2,4,6-trifluorophenyl | —Br | —Cl | 2.77 |
| 58 | t-butyl | —H | 2,4,6-trifluorophenyl | —Br | —Cl | 3.9 |
| 59 | —CH₃ | —CH₃ | 2,4,6-trifluorophenyl | —Br | —Cl | 3.14 |
| 60 | cyclopentyl | —H | 2,4,6-trifluorophenyl | —Br | —Cl | 3.94 |
| 61 | —CH₂—CH₂—S—CH₂—CH₂— | | 2,4,6-trifluorophenyl | —Br | —Cl | 3.72 |
| 62 | cyclopropylmethyl | —H | 2,4,6-trifluorophenyl | —Br | —Cl | 3.5 |
| 63 | 1-cyclohexylethyl | —H | 2,4,6-trifluorophenyl | —Br | —Cl | 4.92. |
| 64 | 2-butyl | —H | 2,4,6-trifluorophenyl | —Br | —Cl | 3.76 |
| 65 | —NH₂ | i-butyl | 2,4,6-trifluorophenyl | —Br | —Cl | 3.67 |
| 66 | —C(CH₃)₂—CF₃ | —H | 2,4,6-trifluorophenyl | —Cl | —Cl | 3.28 |
| 67 | —CH(CF₃)—CH₂—CH₂—CH₂—CH₂— | | 2,4,6-trifluorophenyl | —Cl | —Cl | 3.28 |
| 68 | —O—CH₃ | —CH₃ | 2,4,6-trifluorophenyl | —Br | —Cl | 3.39 |
| 69 | 2-methylbutyl | —H | 2,4,6-trifluorophenyl | —Cl | —Cl | 4.04 |
| 70 | 2-butyl | —H | 2-chlorophenyl | —Cl | —Cl | 3.79 |
| 71 | —O—CH₃ | —CH₃ | 2,4,6-trifluorophenyl | —Cl | —Cl | 3.36 |
| 72 | —CH₂—CH₂—CH═C(CH₃)—CH₂— | | 2,4,6-trifluorophenyl | —Br | —Cl | 4.19 |
| 73 | 2-methylbutyl | —H | 2,4,6-trifluorophenyl | —Br | —Cl | 4.05 |
| 74 | 2-butyl | —H | 2,4,6-trifluorophenyl | —Cl | —Cl | 3.7 |
| 75 | 2-methoxyethyl | —C₂H₅ | 2,4,6-trifluorophenyl | —Br | —Cl | 3.62 |
| 76 | —CH₂—C(CH₃)═CH₂ | —C₂H₅ | 2,4,6-trifluorophenyl | —Br | —Cl | 4.4 |
| 77 | 2-methoxyethyl | —C₂H₅ | 2,4,6-trifluorophenyl | —Cl | —Cl | 3.61 |
| 78 | —CH₂—C(CH₃)═CH₂ | —C₂H₅ | 2,4,6-trifluorophenyl | —Cl | —Cl | 4.4 |
| 79 | —CH₂—CH₂—CH═C(CH₃)—CH₂— | | 2-chlorophenyl | —Cl | —Cl | |
| 80 | 2,2,2-trifluoro-1-methylethyl | —H | 2,4-difluor-6-methoxyphenyl | —O—CH₃ | —Cl | 3.18 |
| 81 | —CH₂—CH₂—CH(CF₃)—CH₂—CH₂— | | 2-chlorophenyl | —Cl | —Cl | 4.22 |
| 82 | —CH₂—CH₂—CH(CH₃)—CH₂—CH₂— | | 2-chlorophenyl | —Cl | —Cl | 4.62 |
| 83 | —CH₂—CH₂—CHF—CH₂—CH₂— | | 2,4,6-trifluorophenyl | —Cl | —Cl | 3.55 |
| 84 | —CH₂—CH₂—N(CH₃)—CH₂—CH₂— | | 2,4,6-trifluorophenyl | —Cl | —Cl | 2.71 |
| 85 | —CH₂—CH₂—CH₂—CH₂—CH(CH₃)— | | 2,4,6-trifluorophenyl | —Cl | —Cl | 4.33 |
| 86 | —CH₂—C(CH₃)═CH₂ | —H | 2,4,6-trifluorophenyl | —Cl | —Cl | 3.37 |
| 87 | —CH₂—CH₂—CH═C(CH₃)CH₂— | | 2,4,6-trifluorophenyl | —Cl | —Cl | 4.21 |
| 88 | —CH₂—CH₂—CH₂—CH(CH₃)—CH₂— | | 2,4,6-trifluorophenyl | —Cl | —Cl | 4.45 |
| 89 | —CH₂—CH₂—CH₂—CH₂—CH₂— | | 2,4,6-trifluorophenyl | —Cl | —Cl | 4.06 |
| 90 | —CH₂—CH₂—CH(CH₃)—CH₂—CH₂— | | 2,4,6-trifluorophenyl | —Cl | —Cl | 4.45 |
| 91 | —CH₂—CH═C(C₂H₅)—CH₂—CH₂— | | 2,4,6-trifluorophenyl | —Cl | —Cl | 4.63 |
| 92 | —CH₂—CH₂—CH₂—CH(CH₃)—CH₂— | | 2-chlorophenyl | —Cl | —Cl | 4.64 |
| 93 | —CH₂—CH═C(C₂H₅)—CH₂—CH₂— | | 2-chlorophenyl | —Cl | —Cl | 4.87 |
| 94 | -i-propyl | —H | 2-chloro-6- | —Cl | —Cl | 3.42 |
| | | | fluorophenyl | | | |
| 95 | -i-propyl | —H | 2,4-difluorophenyl | —Cl | —Cl | 3.31 |
| 96 | 2,2,2-trifluoro-1-methylethyl | —H | 2,4-difluorophenyl | —Cl | —Cl | 3.52 |
| 97 | 2,2,2-trifluoro-1-methylethyl | —H | 2-chlorophenyl | —Cl | —Cl | 3.63 |
| 98 | 2,2,2-trifluoro-1-methylethyl | —H | 2,4,6-trifluorophenyl | —Cl | —Cl | 3:58 |
| 99 | 2,2,2-trifluoro-1-methylethyl | —H | 2,4,6-trifluorophenyl | —Br | —Cl | 3.59 |
| 100 | 2,2,2-trifluoro-1-methylethyl | —H | 2-chloro-6- | —Cl —Cl | 3.6 | |
| | | | fluorophenyl | | | |
| 101 | i-butyl | —H | 2,4-difluorophenyl | —Cl | —Cl | 3.61 |
| 102 | 2-butyl | —H | 2,4-difluorophenyl | —Cl | —Cl | 3.63 |
| 103 | cyclopentyl | —H | 2,4-difluorophenyl | —Cl | —Cl | 3.79 |
| 104 | 2-methoxyethyl | —H | 2,4-difluorophenyl | —Cl | —Cl | 2.82 |
| 105 | cyclopropyl | —H | 2,4-difluorophenyl . | —Cl | —Cl | 3.01 |
| 106 | —CH₂—CF₃ | —H | 2,4-difluorophenyl | —Cl | —Cl | 3.19 |
| 107 | cyclopropylmethyl | —H | 2,4-difluorophenyl | —Cl | —Cl | 3.36 |
| 108 | —CH₂—C(CH₃)═CH₂ | —H | 2,4-difluorophenyl | —Cl | —Cl | 3.3 |
| 109 | —CH(CH₃)—CH₂—CH(CH₃)₂ | —H | 2,4-difluorophenyl | —Cl | —Cl | 4.31 |
| 110 | —CH₂—CH₂—N(CH₃)₂ | —CH₃ | 2,4-difluorophenyl | —Cl | —Cl | 1.51 |
| 111 | propargyl | —CH₃ | 2,4-difluorophenyl | —Cl | —Cl | 3.21 |
| 112 | 1,3-dioxolan-2-ylmethyl | —CH₃ | 2,4-difluorophenyl | —Cl | —Cl | 3.14 |
| 113 | (2-furyl)methyl | —CH₃ | 2,4-difluorophenyl | —Cl | —Cl | 3.69 |
| 114 | i-butyl | —CH₃ | 2,4-difluorophenyl | —Cl | —Cl | 4.08 |
| 115 | 2-methoxyethyl | —CH₃ | 2,4-difluorophenyl | —Cl | —Cl | 3.2 |
| 116 | —CH₂—C(CH₃)═CH₂ | —CH₃ | 2,4-difluorophenyl | —Cl | —Cl | 3.98 |
| 117 | —CH₂—CH₂—N(CH₃)₂ | —C₂H₅ | 2,4-difluorophenyl | —Cl | —Cl | 1.65 |
| 118 | —CH₂—C(CH₃)═CH₂ | —C₂H₅ | 2,4-difluorophenyl | —Cl | —Cl | 4.3 |
| 119 | allyl | —C₂H₅ | 2,4-difluorophenyl | —Cl | —Cl | 3.95 |
| 120 | (2-furyl)methyl | —C₂H₅ | 2,4-difluorophenyl | —Cl | —Cl | 3.98 |
| 121 | (2-tetrahydrofuryl)methyl | —C₂H₅ | 2,4-difluorophenyl | —Cl | —Cl | 3.87 |
| 122 | 2-methoxyethyl | —C₂H₅ | 2,4-difluorophenyl | —Cl | —Cl | 3.51 |
| 123 | —CH₂—COOC₂H₅ | —C₂H₅ | 2,4-difluorophenyl | —Cl | —Cl | 3.61 |
| 124 | n-butyl | —C₂H₅ | 2,4-difluorophenyl | —Cl | —Cl | 4.5 |
| 125 | —C₂H₅ | —C₂H₅ | 2,4-difluorophenyl | —Cl | —Cl | 3.74 |
| 126 | cyclopropylmethyl | n-propyl | 2,4-difluorophenyl | —Cl | —Cl | 4.48 |
| 127 | (2-tetrahydrofuryl)methyl | n-propyl | 2,4-difluorophenyl | —Cl | —Cl | 4.29 |
| 128 | 2-methoxyethyl | n-propyl | 2,4-difluorophenyl | —Cl | —Cl | 3.9 |
| 129 | 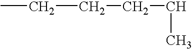 | | 2,4-difluorophenyl | —Cl | —Cl | 3.78 |
| 130 | 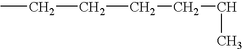 | | 2,4-difluorophenyl | —Cl | —Cl | 4.27 |
| 131 | —CH₂—CH₂—S—CH₂—CH₂ | | 2-chloro-6- | —Cl | —Cl | 3.73 |
| | | | fluorophenyl | | | |
| 132 | —CH₂—CH₂—O—CH₂—CH₂— | | 2-chloro-6- | —Cl | —Cl | 3.02 |
| | | | fluorophenyl | | | |
| 133 | 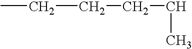 | | 2-chloro-6- fluorophenyl | —Cl | —Cl | 3.92 |
| 134 | —CH₂—CH₂—CH₂—CH₂— | | 2-chloro-6- | —Cl | —Cl | 3.54 |
| | | | fluorophenyl | | | |
| 135 | 2-methoxyethyl | n-propyl | 2-chloro-6- | —Cl | —Cl | 4.06 |
| | | | fluorophenyl | | | |
| 136 | —CH₂—C(CH₃)═CH₂ | —C₂H₅ | 2-chloro-6- | —Cl | —Cl | 4.51 |
| | | | fluorophenyl | | | |
| 137 | allyl | —C₂H₅ | 2-chloro-6- | —Cl | —Cl | 4.16 |
| | | | fluorophenyl | | | |
| 138 | 2-methoxyethyl | —C₂H₅ | 2-chloro-6- | —Cl | —Cl | 3.66 |
| | | | fluorophenyl | | | |
| 139 | i-butyl | —H | 2-chloro-6- | —Cl | —Cl | 3.74 |
| | | | fluorophenyl | | | |
| 140 | 2-butyl | —H | 2-chloro-6- | —Cl | —Cl | 3.75 |
| | | | fluorophenyl | | | |
| 141 | —CH₂—C(CH₃)₃ | —H | 2-chloro-6- | —Cl | —Cl | 4.18 |
| | | | fluorophenyl | | | |
| 142 | cyclopentyl | —H | 2-chloro-6- | —Cl | —Cl | 3.9 |
| | | | fluorophenyl | | | |
| 143 | cyclopropylmethyl | —H | 2-chloro-6- | —Cl | —Cl | 3.49 |
| | | | fluorophenyl | | | |
| 144 | —CH₂—C(CH₃)═CH₂ | —H | 2-chloro-6- | —Cl | —Cl | 3.4 |
| | | | fluorophenyl | | | |
| 145 | —CH(CH₃)—CH₂—CH(CH₃)₂ | —H | 2-chloro-6- | —Cl | —Cl | 4.43 |
| | | | fluorophenyl | | | |
| 146 | allyl | —CH₃ | 2-chloro-6- | —Cl | —Cl | 3.84 |
| | | | fluorophenyl | | | |
| 147 | i-butyl | —CH₃ | 2-chloro-6- | —Cl | —Cl | 4.29 |
| | | | fluorophenyl | | | |
| 148 | 2-methoxyethyl | —CH₃ | 2-chloro-6- | —Cl | —Cl | 3.32 |
| | | | fluorophen yI | | | |
| 149 | —CH₂—C(CH₃)═CH₂ | —CH₃ | 2-chloro-6- | —Cl | —Cl | 4.18 |
| | | | fluorophenyl | | | |
| 150 | —CH₂—CH₂—CH(CF₃)—CH₂—CH₂— | | 2-chloro-6- | —Cl | —Cl | 4.19 |
| | | | fluorophenyl | | | |
| 151 | —CH(CF₃)—CH₂—CH₂CH₂—CH₂— | | 2-chloro-6- | —Cl | —Cl | |
| | | | fluorophenyl. | | | |
| 152 | —NH—CH₂—CH₂—CH₂—CH₂— | | 2,4,6-trifluorophenyl | —N(CH₃)₂ | —Cl | 3.34 |
| 153 | —CH₂—C(CH₃)₃ | —H | 2,4-difluorophenyl | —Cl | —Cl | 4.05 |
| 154 | —CH₂—COOC₂H₅ | —CH₃ | 2,4-difluorophenyl | —Cl | —Cl | 3.33 |
| 155 | allyl | —CH₃ | 2,4-difluorophenyl | —Cl | —Cl | 3.7 |
| 156 | —CH₂—CH(OH)—CH₂—CH₂— | | 2,4-difluorophenyl | —Cl | —Cl | 2.33 |
| 157 | —CH(CF₃)—CH₂—CH₂—CH₂— | | 2,4-difluorophenyl | —Cl | —Cl | 4.09 |
| 158 | —CH₂—CH₂—CH₂—CH₂— | | 2,4-difluorophenyl | —Cl | —Cl | 3.43 |
| 159 | —CH₂—CH₂—CH═CH—CH₂— | | 2,4-difluorophenyl | —Cl | —Cl | 3.76 |
| 160 | 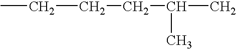 | | 2,4-difluorophenyl | —Cl | —Cl | 4.39 |
| 161 | —CH₂—CH₂—CH(CH₃)—CH₂—CH₂— | | 2,4-difluorophenyl | —Cl | —Cl | 4.4 |
| 162 | —CH₂—CH₂—CH═C(CH₃)—CH₂— | | 2,4-difluorophenyl | —Cl | —Cl | 4.13 |
| 163 | —CH₂—CH₂—CHF—CH₂—CH₂— | | 2,4-difluorophenyl | —CL | —Cl | 3.5 |
| 164 | —CH₂—CH₂—CH(CF₃)—CH₂—CH₂— | | 2,4-difluorophenyl | —Cl | —Cl | 4.07 |
| 165 | —CH₂—CH₂—CH₂—CH₂—CH₂— | | 2,4-difluorophenyl | —Cl | —Cl | 4 |
| 166 | —CH₂—CH₂—O—CH₂—CH₂— | | 2,4-difluorophenyl | —Cl | —Cl | 2.92 |
| 167 | —CH₂—CH₂—S—CH₂—CH₂— | | 2,4-difluorophenyl | —Cl | —Cl | 3.6 |
| 168 | —CH₂—CF₃ | —H | 2-chloro-6- | —Cl | —Cl | 3.25 |
| | | | fluorophenyl | | | |
| 169 | 1,2-dimethylpropyl | H | 2,4,6-trifluorophenyl | —Br | —Cl | 4.02 |
| 170 | 1,2-dimethylpropyl | H | 2-chlorophenyl | —Br | —Cl | 4.18 |
| 171 | 1,2-dimethylpropyl | H | 2,4,6-trifluorophenyl | —Cl | —Cl | 4.01 |
| 172 | 1,2-dimethylpropyl | H | 2-chloro-6- | —Cl | —Cl | 4.09 |
| | | | fluorophenyl | | | |
| 173 | 1,2-dimethylpropyl | H | 2,4-difluorophenyl | —Cl | —Cl | 3.96 |
| 174 | 1,2-dimethylpropyl | H | 2,4,6-trifluorophenyl | —Cl | —Cl | 4.01 |
| 175 | 1,2-dimethylpropyl | H | 2-chlorophenyl | —Cl | —Cl | 4.16 |
| 176 | 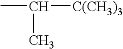 | H | 2,4,6-trifluorophenyl | —Br | —Cl | 4.38 |

*) The logP values were determined in accordance with EEC Directive 79/831 Annex V. A8 by HPLC
(gradient method, acetonitrile/0.1% aqueous phosphoric acid)

Preparation of the Intermediates of the Formula (II)

### Example 177

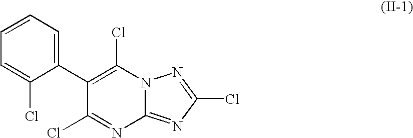 Process b)

63 g (0.41 mol) of phosphorus oxychloride are added to 14.2 g of crude 2-chloro 6-(2-chlorophenyl)-7-hydroxy[1,2,4]triazolo[1,5-a]pyrimidin-5(4H)-one. 5.5 g (26.4 mmol) of phosphorus pentachloride are added a little at a time. The mixture is heated at reflux for 16 hours. After cooling, water is added, and the mixture is extracted 3 times with in each case 100 ml of dichloromethane. The combined organic phases are dried over sodium sulphate and then concentrated under reduced pressure. The residue that remains is purified by silica gel column chromatography using cyclohexane/ethyl acetate (9:1). This gives 2.6 g (17% of theory over 2 steps) of 2,5,7-trichloro-6-(2-chlorophenyl)[1,2,4]triazolo[1,5-a]pyrimidine.

HPLC: logP=3.37

The dihalotriazolopyrimidines of the formula (II) listed in the examples below are also obtained by the method given above.

### Example 178

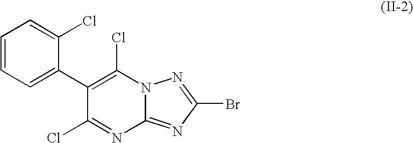

### 2-Bromo-5,7-dichloro-6-(2-chlorophenyl)[1,2,4]triazolo[1,5-a]pyrimidine

HPLC: logP=3.39

### Example 179

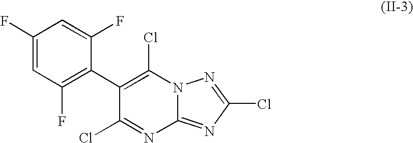

### 2,5,7-Trichloro-6-(2,4,6-trifluorophenyl)[1,2,4]triazolo[1,5-a]pyrimidine

HPLC: logP=3.28

### Example 180

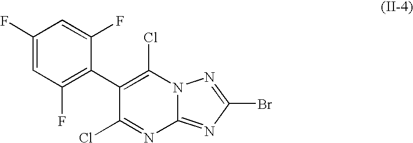

### 2-Bromo-5,7-dichloro-6-(2,4,6-trifluorophenyl)[1,2,4]triazolo[1,5-a]pyrimidine

HPLC: logP=3.27

Preparation of the Intermediates of the Formula (IV)

### Example 181

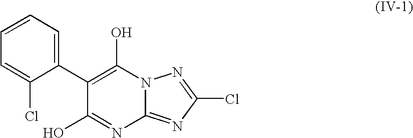 Process c)

5.5 g (46.3 mmol) of 5-chloro-4H-1,2,4-triazol-3-ylamine, 11.2 g (46.3 mmol) of dimethyl-2-(2-chlorophenyl)malonate and 9.4 g (50.9 mmol) of tributylamine are stirred at 180° C. for 16 hours. The methanol formed during the reaction is distilled off continuously. The reaction mixture is concentrated under high vacuum. This gives 14.2 g of crude 2-chloro-6-(2-chlorophenyl)-7-hydroxy[1,2,4]triazolo[1,5-a]pyrimidin-5(4H)-one which is used for the next reaction step without further purification.

The following compounds were obtained analogously:

### Example 182

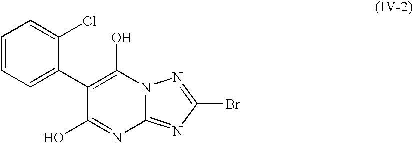

### 2-Bromo-6-(2 chlorophenyl) 7-hydroxy[1,2,4]triazolo[1,5-a]pyrimidin-5(4H)-one

### Example 183

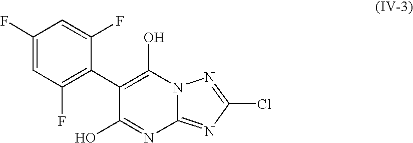

### 2-Bromo-6-(2,4,6-trifluorophenyl)-7-hydroxy[1,2,4]triazolo[1,5-a]pyrimidin-5(4H)-one

### Example 184

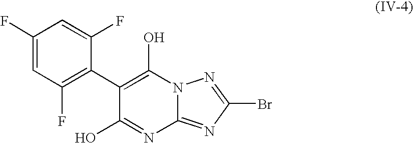

### 2-Chloro-6-(2,4,6-trifluorophenyl)-7-hydroxy[1,2,4]triazolo[1,5-a]pyrimidin-5(4H)-one

The compounds listed in Examples 181-184 can exist both in the dihydroxy form and in the tautomeric keto form. For the compound (IV-1), this can be illustrated by the following formulae. 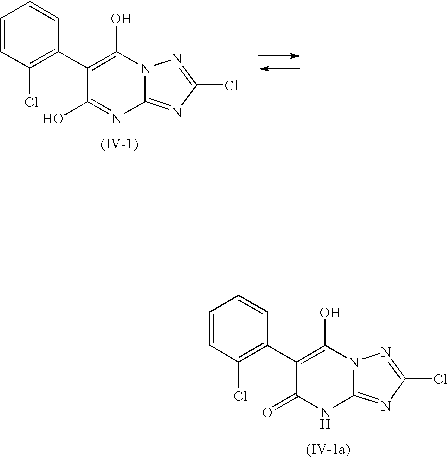
Preparation of the Intermediates of the Formula (VI)

### Example 185

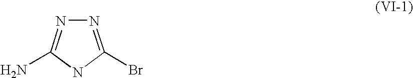

180 ml (3.3 mol) of hydrobromic acid are added to 9.7 g (0.098 mol) of guanazole (1,2,4-triazole-3,5-diamine), and the mixture is cooled to 0-5° C. With stirring, a solution of 8.45 g (0.12 mol) of sodium nitrite in 20 ml of water is slowly added dropwise at this temperature. The reaction mixture is slowly warmed to room temperature and then heated at reflux for 16 h. For work-up, the reaction solution is cooled, poured onto about 11 of ice and made alkaline using half-concentrated aqueous sodium hydroxide solution. The aqueous solution is concentrated under reduced pressure to about 500 ml and extracted in a liquid-liquid extractor with ethyl acetate for a number of days. Removal of the organic solvent gives 12.2 g of 5-bromo-1,2,4-triazole-3-amine in a purity of 80% (yield: 61%).

HPLG: log P=0.37

### USE EXAMPLES

### Example: A

Podosphaera Test (Apple)/Protective

| | |
|---|---|
| Solvents: | 24.5 parts by weight of acetone |
| | 24.5 parts by weight of dimethylacetamide |
| Emulsifier: | 1.0 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of the apple mildew pathogen Podosphaera leucotricha. The plants are then placed in a greenhouse at about 23° C. and a relative atmospheric humidity of about 70%.

Evaluation is carried out 10 days after the inoculation. 0% means an efficacy which corresponds to that of the control, whereas an efficacy of 100% means that no infection is observed.

Active compounds, active compound application rates and tests results are shown in the table below.

TABLE A
Podosphaera test (apple)/protective

| | | |
|---|---|---|
| | Application rate of active | Efficacy |
| Active compound | compound in g/ha | in % |

According to the invention:

| | | |
|---|---|---|
| 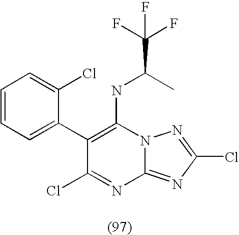 | 100 | 100 |
| 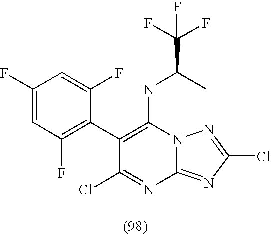 | 100 | 100 |
| 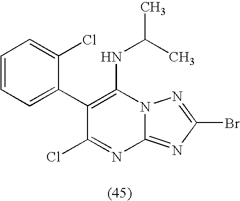 | 100 | 87 |
| 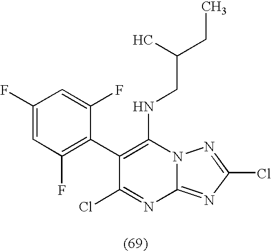 | 100 | 100 |
| 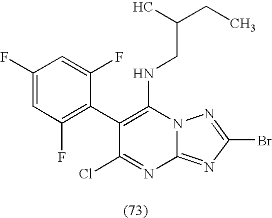 | 100 | 94 |
| 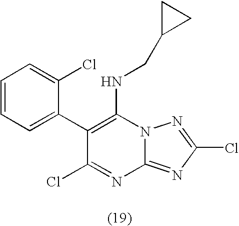 | 100 | 97 |
| 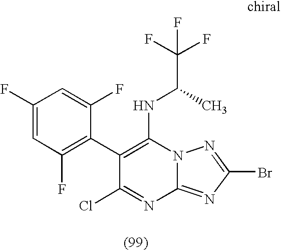 | 100 | 100 |
| 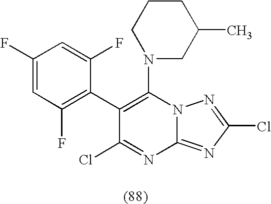 | 199 | 94 |
| 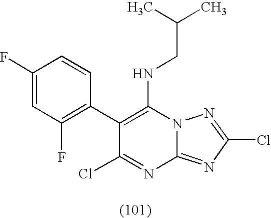 | 100 | 100 |
| 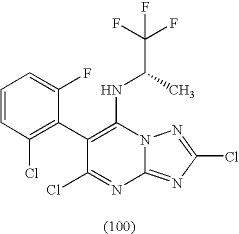 | 100 | 100 |
| 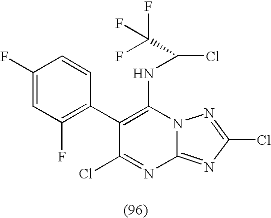 | 100 | 100 |
| 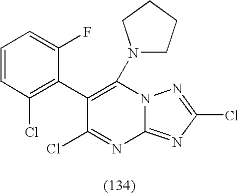 | 100 | 93 |
| 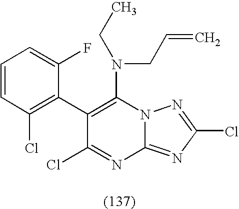 | 100 | 95 |
| 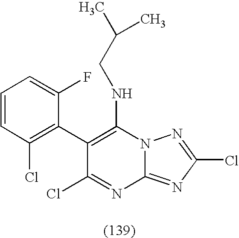 | 100 | 100 |
| 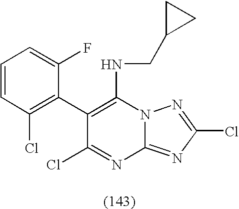 | 100 | 100 |
| 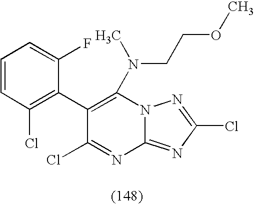 | 100 | 100 |
| 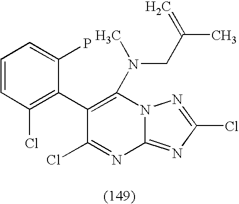 | 100 | 100 |
| 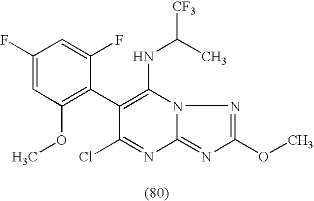 | 100 | 83 |

### Example B

Venturia Test (Apple)/Protective

| | |
|---|---|
| Solvents: | 24.5 parts by weight of acetone |
| | 24.5 parts by weight of dimethylacetamide |
| Emulsifier: | 1.0 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day.

The plants are then placed in a greenhouse at about 21° C. and a relative atmospheric humidity of about 0.90%.

Evaluation is carried out 10 days after the inoculation. 0% means an efficacy which corresponds to that of the control, whereas an efficacy of 100% means that no infection is observed.

Active compounds, active compound application rates and test results are shown in the table below.

TABLE B
Venturia test (apple)/protective

| | | |
|---|---|---|
| | Application rate of active | Efficacy |
| Active compound | compound in g/ha | in % |

According to the invention:

| | | |
|---|---|---|
| 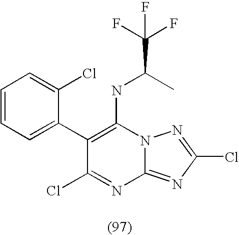 | 100 | 100 |
| 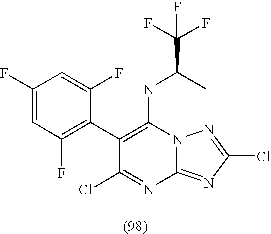 | 100 | 100 |
| 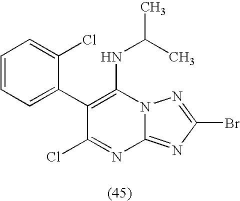 | 100 | 98 |
| 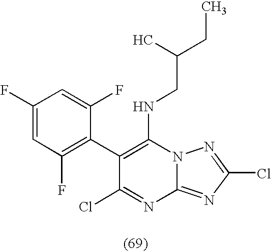 | 100 | 83 |
| 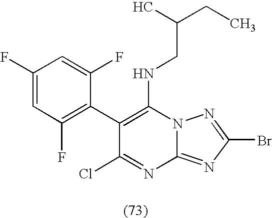 | 100 | 80 |
| 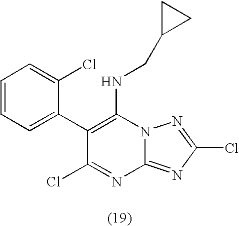 | 100 | 96 |
| 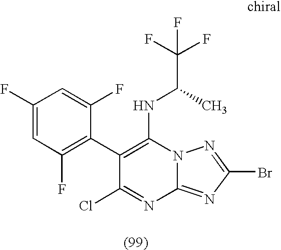 | 100 | 99 |
| 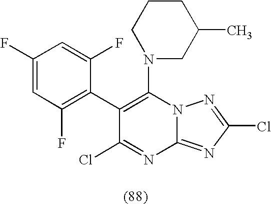 | 100 | 96 |
| 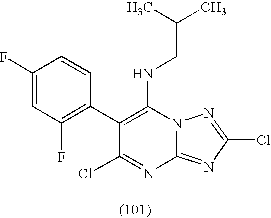 | 100 | 85 |
| 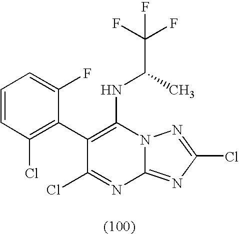 | 100 | 100 |
| 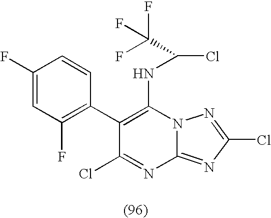 | 100 | 100 |
| 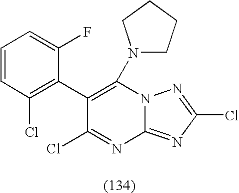 | 100 | 85 |
| 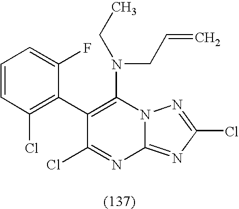 | 100 | 96 |
| 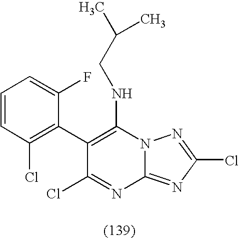 | 100 | 91 |
| 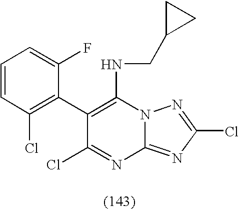 | 100 | 99 |
| 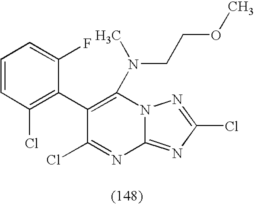 | 100 | 96 |
| 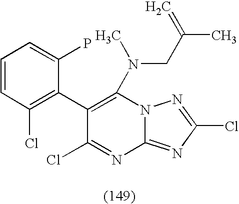 | 100 | 84 |
| 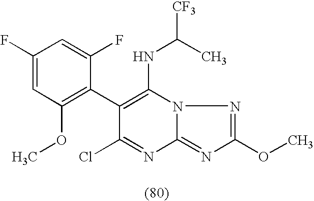 | 100 | 99 |

### Example C

Pyricularia Test (Rice)/Protective

| | |
|---|---|
| Solvent: | 25 parts by weight of N,N-dimethylacetamide |
| Emulsifier: | 0.6 part by weight of alkylaryl polyglycol ether |

To produce a suitable preparation of active compound, 1 part by weight of active compound is mixed with the stated amount of solvent, and the concentrate is diluted with water and the stated amount of emulsifier to the desired concentration.

To test for protective activity, young rice plants are sprayed with the preparation of active compound at the stated application rate. After the spray coating has dried on, the plants are inoculated with an aqueous spore suspension of Pyricularia oryzae. The plants are then placed in a greenhouse at 100% relative atmospheric humidity and at 25° C.

Evaluation is carried out 6 days after the inoculation. 0% means an efficacy which corresponds to that of the control, whereas an efficacy of 100% means that no infection is observed.

Active compounds, active compound application rates and test results are shown in the table below.

TABLE C
Pyricularia test (rice)/protective

| | | |
|---|---|---|
| | Application rate of | |
| | active compound in | Efficacy |
| Active compound | g/ha | in % |

According to the invention:

| | | |
|---|---|---|
| 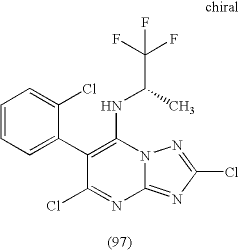 | 250 | 100 |
| 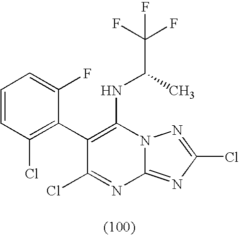 | 250 | 100 |

1. Triazolopyrimidines of the formula 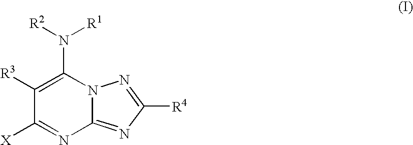
in which
R¹ represents amino, represents in each case optionally substituted alkyl, alkenyl, alkynyl, cycloalkyl, alkoxy, alkenyloxy, alkynyloxy, cycloalkyloxy, alkylamino, dialkylamino, alkenylamino, alkynylamino, cycloalkylamino, N-cycloalkyl-N-alkylamino, alkylideneamino or heterocyclyl, and
R² represents hydrogen or represents in each case optionally substituted alkyl, alkenyl, alkynyl or cycloalkyl, or
R¹ and R² together with the nitrogen atom to which they are attached form an optionally substituted heterocyclic ring,
R³ represents aryl which is optionally mono- to tetrasubstituted,
R⁴ represents halogen, cyano or represents in each case optionally substituted alkoxy or dialkylamino and
X represents halogen.

2. Process for preparing triazolopyrimidines of the formula (1) according to claim 1, characterized in that
(a) dihalotriazolopyrimidines of the formula 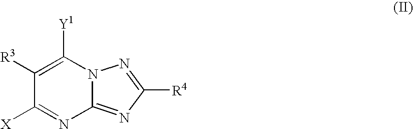
in which
R³, R⁴ and X are as defined above and
Y¹ represents halogen,
are reacted with an amine of the formula 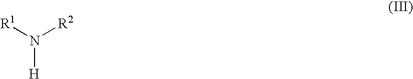
in which
R¹ and R² are as defined above,
if appropriate in the presence of a diluent and if appropriate in the presence of an acid acceptor.

3. Composition for controlling unwanted microorganisms, characterized in that it comprises at least one triazolopyrimidine of the formula (I) according to claim 1, in addition to extenders and/or surfactants.

4. Use of triazolopyrimidines of the formula (I) according to claim 1 for controlling unwanted microorganisms.

5. Method for controlling unwanted microorganisms, characterized in that triazolopyrimidines of the formula (I) according to claim 1 are applied to the unwanted microorganisms and/or their habitat.

6. Process for preparing compositions for controlling unwanted microorganisms, characterized in that triazolopyrimidines of the formula (I) according to claim 1 are mixed with extenders and/or surfactants.

7. Dihalotriazolopyrimidines of the formula 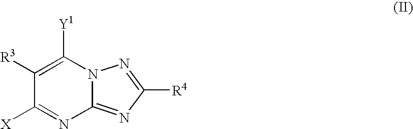
in which
R³, R⁴ and X are as defined above and
Y¹ represents halogen.

8. Process for preparing dihalotriazolopyrimidines of the formula (U) according to claim 7, characterized in that
(b) dihydroxytriazolopyrimidines of the formula 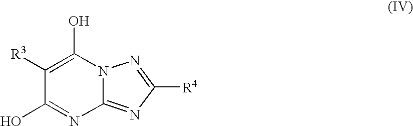
in which
R³ and R⁴ are as defined above
are reacted with halogenating agents, if appropriate in the presence of a diluent.

9. Dihydroxytriazolopyrimidines of the formula 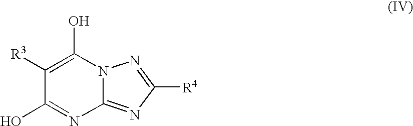
in which
R³ and R⁴ are as defined above.

10. Process for preparing dihydroxytriazolopyrimidines of the formula (IV) according to claim 9, characterized in that
(c) arylmalonic esters of the formula 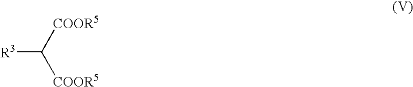
in which
R³ is as defined above and
R⁵ represents alkyl having 1 to 4 carbon atoms
are reacted with aminotriazoles of the formula 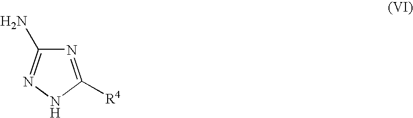
in which
R⁴ is as defined above,
if appropriate in the presence of a diluent and if appropriate in the presence of an acid binder.

11. Aminotriazoles of the formula 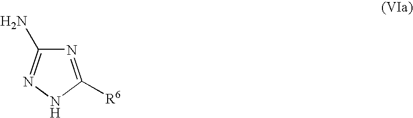
in which
R⁶ represents cyano or bromine.

12. Process for preparing aminotriazoles of the formula (VI) according to claim 11, characterized in that diaminotriazole of the formula 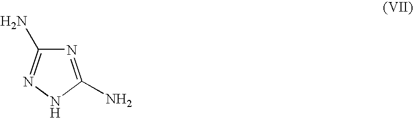
is initially diazotized and then reacted with a brominating agent or a cyanating agent, if appropriate in the presence of a diluent and if appropriate in the presence of further reaction auxiliaries.
